(19) United States
(12) Patent Application Publication
(10) Pub. No.: US 2025/0152626 A1
(43) Pub. Date: May 15, 2025

(54) CONSTRUCTION AND APPLICATION OF ENHANCED NEUTROPHIL DRUG

(71) Applicant: China Pharmaceutical University, Nanjing (CN)

(72) Inventors: Can Zhang, Nanjing (CN); Caoyun Ju, Nanjing (CN); Yijun Chen, Nanjing (CN); Qifan Hu, Nanjing (CN)

(73) Assignee: China Pharmaceutical University, Nanjing (CN)

(21) Appl. No.: 18/931,268

(22) Filed: Oct. 30, 2024

(30) Foreign Application Priority Data
Nov. 9, 2023 (CN) 202311490898.0

(51) Int. Cl. A61K 35/15; A61K 31/337; A61K 31/7028; A61P 31/10; A61P 35/00; C12N 5/0787

(52) U.S. Cl. CPC A61K 35/15; A61K 31/337; A61K 31/7028; A61P 31/10; A61P 35/00; C12N 5/0642; C12N 2501/22; C12N 2501/24; C12N 2501/25; C12N 2501/51; C12N 2501/734; C12N 2523/00

(57) ABSTRACT

The present disclosure relates to construction and an application of an enhanced neutrophil drug, specifically to a construction method for obtaining an enhanced neutrophil, including: performing induction culture of a neutrophil in a medium including any one or more of component A, component B and component C, where the component A includes one or more of IFNγ, GM-CSF, G-CSF, IFN-β, IFN-α, TNFα, LPS, IL1β, IL6, MCP-1, IL-23, BCG, or β-glucan; the component B includes one or more of durvalumab, avelumab, atezolizumab, ipilimumab, or tremelimumab; and the component C includes one or more of Q-VD-Oph, DFO, Hsp70, NAC, or Nec-1. The construction method provided by the present disclosure prolongs the in vitro lifespan of the neutrophil, and enhances tumor-killing activity, an immune activation ability and/or an anti-infection ability.

### CROSS-REFERENCE TO RELATED APPLICATION

The present disclosure claims priority to Chinese Patent Application No. 202311490898.0, filed on Nov. 9, 2023, the entire content of which is incorporated herein by reference.

### TECHNICAL FIELD

The present disclosure relates to the field of biotechnology, and in particular to construction and an application of an enhanced neutrophil drug.

### BACKGROUND

Neutrophils are the most abundant white blood cells in the human blood, serving as the body's first line of defense against foreign substances. In addition to effectively killing pathogens, neutrophils have also demonstrated unique advantages in tumor treatment, including: (1) neutrophils can quickly respond to inflammatory factors released from tumor sites, actively approach tumor sites, and can extravasate from tumor blood vessels and penetrate into the deep layers of tumors, highly infiltrating in various tumors¹; (2) neutrophils themselves have the ability to kill tumors, which can kill tumor cells by releasing reactive oxygen species (ROS), nitric oxide (NO), antibody-dependent cell-mediated cytotoxicity (ADCC), antibody-dependent cell-mediated phagocytosis (ADCP), etc.; and (3) neutrophils have immune regulatory effects and can recruit and activate T cells or NK cells by releasing cytokines². Based on this, multiple studies have reported neutrophil-based anti-tumor therapies in recent years, which are summarized as follows:

(1) Researchers used the infusion of white blood cells from healthy unrelated donors to treat solid tumors (phase I/II, NCT00900497). White blood cells from 4-5 donors were collected by apheresis and infused into patients at a dose of 2×10¹⁰⁻¹¹ cells/time, which was expected to exert a good therapeutic effect. For leukocyte transfusion therapy, it is necessary to obtain a sufficient number of neutrophils from donors before transferring them to cancer patients. This therapy has the following drawbacks: 1) neutrophils have a short in vitro lifespan (usually less than 24 hours) and are difficult to store; and 2) the tumor-killing efficacy of donor-derived neutrophils varies among individuals, leading to inconsistent treatment results, and most neutrophils have an insufficient tumor-killing ability.

(2) Researchers showed that the tumor-killing efficacy of neutrophils was genetically defined. Therefore, they screened healthy individuals with neutrophils with high tumor-killing activity, isolated hematopoietic stem cells from these donors, and differentiated them into neutrophils with similar high tumor-killing activity using some cytokines or vitamins. Researchers provided cultures of hematopoietic stem cells differentiating into neutrophils, including hematopoietic stem cells, granulocyte-macrophage colony-stimulating factors (GM-CSF), granulocyte colony-stimulating factors (G-CSF), growth hormones, serotonin, vitamin C, vitamin D, glutamine (Gln), arachidonic acid, AGE-albumin, interleukins (IL), tumor necrosis factor (TNF) a, Flt-3 ligands, thrombopoietin, and fetal bovine serum (CN 111278968 A). The method of using donors with neutrophils with high tumor-killing activity as sources of hematopoietic stem cells, and then differentiating hematopoietic stem cells into neutrophils with similar high tumor-killing activity for tumor treatment has the following drawbacks: 1) it requires large-scale screening of the population to find donors of neutrophils with high tumor-killing activity, and the number of donors is limited; 2) inducing differentiation of hematopoietic stem cells into neutrophils requires a long induction cycle and a large number of cytokines, which is time-consuming and expensive; and 3) the induced neutrophils have purity of less than 80%, including monocytes and macrophages that also express CD11b and CD15, and mixed with 4%-10% hematopoietic stem cells. There is a risk of transfusion-related acute lung injury caused by monocytes and graft-versus-host disease caused by hematopoietic stem cells.

(3) Based on the plasticity of neutrophils³, researchers used different cytokines to stimulate neutrophils to exert a certain degree of anti-tumor effects. For example, using a single inducer interferon (IFN)-7 can prolong the lifespan of neutrophils. 500 U/mL IFNγ can maintain the viability of neutrophils at over 80% to 48 h⁴ and can upregulate CD64, ICAM-1 and HLA-DR, which may be beneficial for tumor-killing⁵. Researchers also optimized the concentration of a single inducer IFNα and obtained an induction method to enhance neutrophil activity, which can transform N2 (pro-tumor) neutrophils into N1 (anti-tumor) neutrophils, thereby enhancing the tumor-killing effect (CN110066766A). In early but not advanced non-small cell lung cancer, 50 pg/mL to 20 ng/mL IFNγ and 50 pg/mL GM-CSF have been shown to drive neutrophil differentiation towards APC-like MHCII neutrophils expressing co-stimulatory molecules OX40 ligand, CD86 and 4-1BB ligand, and these APC-like MHCII neutrophils can stimulate and enhance anti-tumor effector T cell responses. In addition, researchers pre-stimulated neutrophils with 1 nM Phorbol 12-myristate 13-acetate (PMA) for 30 minutes, and then added an additional concentration of 40 μg/mL of oxidized low-density lipoprotein (ox-LDL) for 12-h stimulation to obtain neutrophils with antigen-presenting cell phenotypes, which can promote T cell proliferation and produce IFN-7 (CN114045261A). There are currently limitations to the technology of cytokine induction of neutrophils against tumors: 1) there are time- and concentration-dependent differences in the effects of different cytokines on neutrophils, but no systematic studies have been conducted so far; 2) the effects of cytokines on neutrophils are multifaceted. Although they can enhance the tumor-killing effect, they may also cause immunosuppression. The anti-tumor effect of neutrophils induced solely by cytokines in vivo has not been effectively validated, and the therapeutic effect is doubtful; and 3) considering that clinical application requires longer neutrophil lifespan and higher tumor-killing efficacy, there is currently a lack of combined inducers for both.

(4) Our team used the phagocytosis of neutrophils to construct neutrophil drugs for tumor treatment in the early stage (CN104225609B). Although the neutrophil drug delivery system can significantly improve the targeting effect and therapeutic effect of drugs, the anti-tumor effect of neutrophils, as drug carriers, is not sufficient to effectively inhibit tumor growth.

### REFERENCES

1 Jaillon, S. et al. Neutrophil diversity and plasticity in tumour progression and therapy. Nature Reviews Cancer 20, 485-503 (2020).
2 Ustyanovska Avtenyuk, N. V., Nienke Bremer, Edwin Wiersma, Valerie R. The Neutrophil: The underdog that packs a punch in the fight against cancer. International Journal of Molecular Sciences 21, 7820-7878 (2020).
3 Giese, M. A., Hind, L. E. & Huttenlocher, A. Neutrophil plasticity in the tumor microenvironment. Blood 133, 2159-2167 (2019).
4 Colotta F, R. F., Polentarutti N, Sozzani S, Mantovani A. Modulation of granulocyte survival and programmed cell death by cytokines and bacterial products. Blood 80, 2012-2020 (1992).
5 Ricardo, G.-B., Tarik, E. N., S. Hackert & Felix, A. R. S., A. Jelinsky Olha, Halyabar Alexandra. Ageing and interferon gamma response drive the phenotype of neutrophils in the inflamed joint. Annals of the Rheumatic Diseases 81, 805-814 (2022).

### SUMMARY

Given the drawbacks of the prior art mentioned above, the purpose of the present disclosure is to provide a construction method for obtaining an enhanced neutrophil to prolong the in vitro life of the neutrophil, and enhance its tumor-killing activity and immune activation ability.

To solve the above technical problems, the technical solution adopted by the present disclosure is as follows:

A first purpose of the present disclosure is to provide a construction method for obtaining an enhanced neutrophil, including: performing induction culture of a neutrophil in a medium including any one or more of component A, component B and component C, where the component A includes one or more of IFNγ, GM-CSF, G-CSF, IFN-β, IFN-α, TNFα, LPS, IL1β, IL6, MCP-1, IL-23, BCG, or β-glucan;
the component B includes one or more of durvalumab, avelumab, atezolizumab, ipilimumab, or tremelimumab; and
the component C includes one or more of Q-VD-Oph, DFO, Hsp70, NAC, or Nec-1.

Preferably, the component A is selected from IFNγ and/or GM-CSF.

Preferably, the component B is selected from durvalumab.

Preferably, the component C is selected from Q-VD-Oph.

Preferably, a concentration of the component A is 1-200 ng/mL, preferably 1-100 ng/mL, more preferably 1-50 ng/mL, particularly preferably 1-30 ng/mL, and most preferably 20 ng/mL.

Preferably, a concentration of the component B is 0.1-10 μg/mL, preferably 0.5-8 μg/mL, more preferably 0.5-5 μg/mL, particularly preferably 1-3 μg/mL, and most preferably 2 μg/mL.

Preferably, a concentration of the component C is 1-100 μM, preferably 10-80 μm, more preferably 10-60 μM, particularly preferably 20-60 μM, and most preferably 50 μM.

Preferably, the medium is 1640 medium or DMEM medium.

Preferably, the method includes incubating the neutrophil in the medium in an aerobic or anaerobic environment.

Preferably, a temperature of the incubating is controlled at 35-40° C., preferably 36-40° C., and more preferably 37° C.

Preferably, time of the incubating is controlled to be 2-48 h, preferably 2-36 h, more preferably 4-24 h, and most preferably 4-18 h.

Preferably, the incubating is controlled to be carried out under a condition of 5% CO₂.

Preferably, the neutrophil is derived from a healthy individual or a tumor patient.

A second purpose of the present disclosure is to provide an enhanced neutrophil obtained by the construction method according to any one of the above.

A third purpose of the present disclosure is to provide a drug composition, including the enhanced neutrophil obtained by the construction method according to any one of the above or the enhanced neutrophil according to the above.

Preferably, the drug composition further includes a nano-drug.

Further preferably, the nano-drug is selected from an anti-tumor drug, an immunomodulatory drug and an antibacterial drug.

Further preferably, the anti-tumor drug is selected from paclitaxel, doxorubicin, 5-fluorouracil, and camptothecin.

Further preferably, the immunomodulatory drug is selected from a STING modulator, a TGF-β modulator and a HIF-1α modulator.

Further preferably, the antibacterial drug is selected from amphotericin B, colistin, azithromycin, clavulanic acid, ampicillin, ciprofloxacin, and vancomycin.

A fourth objective of the present disclosure is to provide a method for preparing a drug composition, including: mixing the enhanced neutrophil obtained by the construction method according to any one of the above or the enhanced neutrophil according to the above with a nano-drug, and then incubating in an aerobic or anaerobic environment.

Preferably, a temperature of the incubating is controlled at 35-40° C., preferably 36-40° C., and more preferably 37° C.

Preferably, time of the incubating is controlled to be 0.1-12 h, preferably 0.2-10 h, more preferably 0.2-6 h, and most preferably 0.2-3 h.

Preferably, the incubating is controlled to be carried out under a condition of 5% CO₂.

A fifth objective of the present disclosure is to provide an application of the enhanced neutrophil obtained by the construction method according to any one of the above, the enhanced neutrophil according to the above, the drug composition according to the above, or the drug composition obtained by the preparation method according to the above for prevention and/or treatment of a tumor, an infection or an immune disease.

Compared with the prior art, the present disclosure has the following technical effects:

The enhanced neutrophil obtained by the construction method provided by the present disclosure has a longer lifespan and stronger anti-tumor and immunomodulatory abilities, and has the advantages of a simple operation method, a mild condition, short time consumption, etc.

The enhanced neutrophil drug composition obtained by the construction method provided by the present disclosure can jointly exert the therapeutic effects of the enhanced neutrophil and the nano-drug, and can break through the limitation that the neutrophil is only used as a drug carrier.

### BRIEF DESCRIPTION OF THE DRAWINGS

FIG. 1 is a flow chart of a construction process of enhanced neutrophils and drug compositions in the present disclosure;

FIG. 2 is a bar graph showing effects of different concentrations of single inducers on the in vitro lifespan of neutrophils in Example 1 of the present disclosure;

FIG. 3 is a bar graph showing effects of different combined inducers on the in vitro lifespan of neutrophils in Example 2 of the present disclosure;

FIG. 4 is a bar graph showing effects of different combined inducers on the in vitro tumor-killing ability of neutrophils in Example 3 of the present disclosure;

FIG. 5 is a graph showing viability comparison of neutrophils treated with a combined inducer and single inducers in Example 4 of the present disclosure, where the control group is untreated neutrophils;

FIG. 6 shows killing abilities of enhanced neutrophils against different tumor cells in Example 5 in the present disclosure, where HELA: human cervical cancer cells; A549: human non-small cell lung cancer cells; PANC-1: human pancreatic cancer cells; MCF7: human breast cancer cells; T24: human bladder transitional cell carcinoma cells; SW480: human colon cancer cells; A-375: human malignant melanoma cells; and U251: human glioma cells;

FIG. 7 is a bar graph showing tumor cell-killing abilities of enhanced neutrophils induced with neutrophils from different sources in Example 6, where young people are aged 18-25 years, middle-aged people are aged 30-45 years, and elderly people are aged 60 years and above;

FIG. 8 is a bar graph showing the ability of enhanced neutrophils to recruit and activate T cells in Example 7 in the present disclosure;

FIG. 9 is a bar graph showing the ability of enhanced neutrophils of the present disclosure to recruit and activate NK cells in Example 7 in the present disclosure;

FIG. 10 is a survival curve of glioma mice treated with a drug composition in Example 8 in the present disclosure;

FIG. 11 is a survival curve of mice with triple-negative breast cancer lung metastasis treated with a drug composition in Example 9 in the present disclosure; and

FIG. 12 shows unit values of colony formation in the brain of mice with infiltrating fungal infection treated with a drug composition in Example 10 in the present disclosure.

### DETAILED DESCRIPTION

In order to make the technical solution and beneficial effects of the present disclosure more obvious and understandable, the following will provide a detailed explanation by listing specific embodiments. The drawings are not necessarily drawn to scale, and local features can be enlarged or reduced to display the details of local features more clearly; and unless otherwise defined, the technical and scientific terms used herein have the same meanings as technical and scientific terms used in the technical field to which the present disclosure belongs.

In response to the limitation that neutrophils only serve as delivery carriers in neutrophil drug delivery systems and cannot effectively exert their anti-tumor effects, inventors propose a construction method of an enhanced neutrophil after extensive studies and experiments (principle shown in FIG. 1), specifically including performing induction culture of a neutrophil in a medium including any one or more of component A, component B and component C. Furthermore, the component A is a cytokine, including but not limited to IFN-7, GM-CSF, G-CSF, IFN-β, IFN-α, lipopolysaccharide (LPS), interleukin (IL), monocyte chemoattractant protein-1 (MCP-1), Bacillus Calmette-Guérin (BCG), TNFα, β-glucan, and other cytokines; the component B is an immune checkpoint inhibitor, including but not limited to durvalumab, avelumab, atezolizumab, ipilimumab, and tremelimumab; and the component C is an anti-apoptotic drug, including but not limited to broad-spectrum caspase inhibitor (Q-VD-Oph), deferoxamine (DFO), heat shock protein 70 (HSP70), N-acetylcysteine (NAC), and necrotic apoptosis inhibitor necrostatin-1 (Nec-1). The enhanced neutrophil obtained by the construction method provided by the present disclosure has the advantages of a longer lifespan and stronger anti-tumor and immunomodulatory abilities.

A first purpose of the present disclosure is to provide a construction method for obtaining an enhanced neutrophil, including: performing induction culture of a neutrophil in a medium including any one or more of component A, component B and component C, where the component A includes one or more of IFNγ, GM-CSF, G-CSF, IFN-β, IFN-α, TNFα, LPS, IL1β, IL6, MCP-1, IL-23, BCG, or β-glucan;
the component B includes one or more of durvalumab, avelumab, atezolizumab, ipilimumab, or tremelimumab; and
the component C includes one or more of Q-VD-Oph, DFO, Hsp70, NAC, or Nec-1. The present disclosure obtains the enhanced neutrophil with a long lifespan, high tumor-killing activity and a high immune activation ability by optimizing components of a combined inducer, and an action concentration and action time of each component. The construction method provided by the present disclosure prolongs the in vitro lifespan of the neutrophil and facilitates quality control.

In some embodiments, the method includes: performing induction culture of a neutrophil in a medium including component A, component B and component C.

In some embodiments, the component A is selected from IFNγ and/or GM-CSF.

In some embodiments, the component B is selected from durvalumab.

In some embodiments, the component C is selected from Q-VD-Oph.

In some embodiments, a concentration of the component A is 1-200 ng/mL, such as 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, 100 ng/mL, 110 ng/mL, 120 ng/mL, 130 ng/mL, 140 ng/mL, 150 ng/mL, 160 ng/mL, 170 ng/mL, 180 ng/mL, 190 ng/mL, etc.

In some embodiments, the concentration of the component A is 1-100 ng/mL.

In some embodiments, the concentration of the component A is 1-50 ng/mL.

In some embodiments, the concentration of the component A is 1-30 ng/mL.

In some embodiments, the concentration of the component A is 20 ng/mL.

In some embodiments, a concentration of the component B is 0.1-10 μg/mL, such as 0.5 g/mL, 1.0 μg/mL, 1.5 μg/mL, 2.0 μg/mL, 2.5 μg/mL, 3.0 μg/mL, 3.5 μg/mL, 4.0 μg/mL, 4.5 g/mL, 5.0 μg/mL, 5.5 μg/mL, 6.0 μg/mL, 6.5 μg/mL, 7.0 μg/mL, 7.5 μg/mL, 8.0 μg/mL, 8.5 g/mL, 9.0 μg/mL, 9.5 μg/mL, 10 μg/mL, etc.

In some embodiments, the concentration of the component B is 0.5-8 μg/mL.

In some embodiments, the concentration of the component B is 0.5-5 μg/mL.

In some embodiments, the concentration of the component B is 1-3 μg/mL.

In some embodiments, the concentration of the component B is 2 μg/mL.

In some embodiments, a concentration of the component C is 1-100 μM, such as 5 μM, M, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, etc.

In some embodiments, the concentration of the component C is 10-80 μM.

In some embodiments, the concentration of the component C is 10-60 μM.

In some embodiments, the concentration of the component C is 20-60 μM.

In some embodiments, the concentration of the component C is 50 μM. The present disclosure obtains the enhanced neutrophil with a long lifespan, high tumor-killing activity and a high immune activation ability by optimizing components of a combined inducer, and an action concentration and action time of each component. The construction method provided by the present disclosure prolongs the in vitro lifespan of the neutrophil and facilitates quality control.

In the present disclosure, the medium is selected from Dulbecco's Modified Eagle Medium (DMEM) and RPMI-1640 medium. Commercially available DMEM and RPMI-1640 media, which are well-known in the art, are adopted.

In some embodiments, the medium contains 0.5-5 wt % penicillin streptomycin and 5-20 vol % serum or plasma.

In some embodiments, the medium is 1640 medium or DMEM medium containing 0.5-2 wt % penicillin streptomycin and 5-15 vol % serum.

In some embodiments, the medium is 1640 medium or DMEM medium containing 1 wt % penicillin streptomycin and 10 vol % serum.

In some embodiments, the medium can also be a serum-free medium.

In some embodiments, the method includes: incubating the neutrophil in the medium in an aerobic or anaerobic environment. The construction method provided by the present disclosure has simple and gentle operation and short time consumption.

In some embodiments, a temperature of the incubating is controlled at 35-40° C., such as 36° C., 37° C., 38° C., 39° C., etc.

In some embodiments, the temperature of the incubating is controlled at 36-40° C.

In some embodiments, the temperature of the incubating is controlled at 37° C.

In some embodiments, time of the incubating is controlled to be 2-48 hours, such as 2 h, 4 h, 6 h, 10 h, 12 h, 18 h, 20 h, 24 h, 32 h, 36 h, 48 h, etc. The present disclosure obtains the enhanced neutrophil with a long lifespan, high tumor-killing activity and a high immune activation ability by optimizing components of a combined inducer, and an action concentration and action time of each component.

In some embodiments, the time of the incubating is controlled to be 2-36 h.

In some embodiments, the time of the incubating is controlled to be 4-24 h.

In some embodiments, the time of the incubating is controlled to be 4-18 h.

In some embodiments, the incubating is controlled to be carried out under a condition of 5% CO₂.

In some embodiments, the neutrophil is derived from a healthy individual or a tumor patient.

The construction method for obtaining an enhanced neutrophil provided by the present disclosure is not limited by the activity of a donor neutrophil, and can be derived from a neutrophil of individuals with different ages and genders, healthy individuals, and even patients, expanding a donor source.

A second purpose of the present disclosure is to provide an enhanced neutrophil obtained by the construction method according to any one of the above.

The enhanced neutrophil provided by the present disclosure can be further prepared into a neutrophil combination drug with a chemotherapeutic drug, an immunomodulator or an anti-infection drug, etc., which can not only respond to chemokines released by the tumor microenvironment, but also quickly enrich and target to the tumor in situ and metastatic tumor or infection sites, thereby increasing the enrichment quantity of loaded drugs in the target sites and improving the drug efficacy thereof; at the same time, the neutrophil in the disease microenvironment can also exert its own tumor-killing, immune activation or bactericidal effects, increasing the intratumoral infiltration and anti-tumor efficacy or bactericidal ability of T cells and NK cells; and finally, a combined therapeutic effect is exerted, and the therapeutic effect for solid tumors and infections is significantly improved. At the same time, the doses of both can be relatively reduced to better meet clinical needs.

A third purpose of the present disclosure is to provide a drug composition, including the enhanced neutrophil obtained by the construction method according to any one of the above or the enhanced neutrophil according to the above.

In some embodiments, the drug composition further includes a nano-drug.

In some embodiments, the nano-drug is selected from an anti-tumor drug, an immunomodulatory drug and an antibacterial drug.

In some embodiments, the anti-tumor drug is selected from paclitaxel, doxorubicin, 5-fluorouracil, and camptothecin.

In some embodiments, the immunomodulatory drug is selected from a STING modulator, a TGF-β modulator and a HIF-la modulator. The “super” neutrophil combination drug constructed by the present disclosure has a synergistic therapeutic effect (principle shown in FIG. 1).

In some embodiments, the antibacterial drug is selected from amphotericin B, colistin, azithromycin, clavulanic acid, ampicillin, ciprofloxacin, and vancomycin.

A fourth objective of the present disclosure is to provide a method for preparing a drug composition, including: mixing the enhanced neutrophil obtained by the construction method according to any one of the above or the enhanced neutrophil according to the above with a nano-drug, and then incubating in an aerobic or anaerobic environment.

In some embodiments, a temperature of the incubating is controlled at 35-40° C., such as 36° C., 37° C., 38° C., 39° C., etc.

In some embodiments, the temperature of the incubating is controlled at 36-40° C.

In some embodiments, the temperature of the incubating is controlled at 37° C.

In some embodiments, time of the incubating is controlled to be 0.1-12 h, such as 0.1 h, 0.2 h, 0.5 h, 1 h, 2 h, 3 h, 4 h, 5 h, 6 h, 7 h, 8 h, 9 h, etc.

In some embodiments, the time of the incubating is controlled to be 0.2-10 h.

In some embodiments, the time of the incubating is controlled to be 0.2-6 h.

In some embodiments, the time of the incubating is controlled to be 0.2-3 h.

In some embodiments, the incubating is controlled to be carried out under a condition of 5% CO₂.

A fifth objective of the present disclosure is to provide an application of the enhanced neutrophil obtained by the construction method according to any one of the above, the enhanced neutrophil according to the above, the drug composition according to the above, or the drug composition obtained by the preparation method according to the above in preventing and/or treating of a tumor, an infection or an immune disease.

Preferably, the tumor includes but is not limited to breast cancer, cervical cancer, non-small cell lung cancer, pancreatic cancer, bladder cancer, colon cancer, melanoma, gliomas, etc.

Preferably, the infection includes but is not limited to a viral infection, a bacterial infection, a fungal infection, a parasitic infection, a bacterial infection or a fungal infection accompanying agranulocytosis due to chemotherapy, an infiltrating fungal infection, sepsis, Corona Virus Disease 2019, etc.

Preferably, the immune disease includes but is not limited to rheumatoid arthritis, systemic lupus erythematosus, allergic asthma, etc.

### Definition

In the present disclosure, the term “comprising” or “comprises” is used with respect to combinations, methods and corresponding components thereof that can be used in implementations, and remains an open term that includes unspecified elements, regardless of whether the elements are useful or not. Those skilled in the art should understand that in general, the terms used herein are intended to be “open” terms (for example, the term “comprising” should be construed as “comprising but not limited to”, the term “having” should be construed as “at least having”, the term “including” should be construed as “including but not limited to”, etc.).

In the present disclosure, the term “cytokine” is a molecule produced by various cells (such as monocytes and macrophages) which acts as a mediator of an inflammatory process.

In specific and preferred implementations, the cytokine includes but is not limited to IFN-7, GM-CSF, G-CSF, IFN-β, IFN-α, lipopolysaccharide (LPS), interleukin (IL), monocyte chemoattractant protein-1 (MCP-1), Bacillus Calmette-Guérin (BCG), TNFα, β-glucan, etc.

In the present disclosure, the term “immune checkpoint inhibitor” refers to a drug that can block certain proteins produced by certain types of immune cells (such as T cells) and certain cancer cells. These proteins help control immune responses and can prevent T cells from killing cancer cells. When these proteins are blocked, the brakes on the immune system are released, allowing T cells to be capable of better killing cancer cells. Examples of the immune checkpoint inhibitor include but are not limited to: a PD-1 inhibitor, a CTLA-4 inhibitor, a LAG-3 inhibitor, a TIM-3 inhibitor, a BTLA inhibitor, a PD-L1 inhibitor, a PD-L2 inhibitor, a B7-1 inhibitor, a B7-2 inhibitors, a galectin-9 inhibitor, and a HVEM inhibitor. The immune checkpoint inhibitor can be a small molecule, a peptide, a protein (e.g., an antibody), a nucleic acid, etc.

In the present disclosure, the “PD-L1 inhibitor” refers to an antibody or other molecule that inhibits the function of programmed death ligand 1 (PDL1). In a specific implementation, the antibody is durvalumab, avelumab and atezolizumab.

In the present disclosure, the “CTLA4 inhibitor” refers to an antibody or other molecule that inhibits the function of cytotoxic T lymphocyte-associated antigen 4 (CTLA4). In a specific implementation, the antibody is ipilimumab and tremelimumab.

In the present disclosure, the term “TGF-β” is a type of growth factor that is produced in almost all cells from e.g. kidneys, bone marrow, platelets, etc. There are five subtypes of TGF-β (β1 to β5). It is known that TGF-β promotes the proliferation of osteoblasts and the synthesis and proliferation of connective tissue such as collagen, and has inhibitory effects on the proliferation of epithelial cells and osteoclasts. The term “TGF-β modulator” refers to a substance that blocks or inhibits, for example, binding of TGF-β to TGF-β receptors, and is an agent that binds to TGF-β to form a complex for neutralizing TGF-β activity. The TGF-β modulator includes but is not limited to one or more of A83-01, SB-431542, SB-505124, SB-525334, SD-208, LY-36494, and SJN-2511.

In the present disclosure, the term “HIF-1α” refers to a hypoxia-inducible factor, and in cells, the HIF signaling cascade is influenced by the state of hypoxia. In a state of hypoxia, cells are generally allowed to continue to differentiate. However, the hypoxic state promotes angiogenesis, which is very important for the vascular system in the embryo and cancer tumors. The term “HIF-1α modulator” refers to a compound or a composition capable of being used to inhibit HIF-1α activity. The HIF-1α modulator includes but is not limited to one or more of BAY87-2243, KC7F2, LW 6, and PX-478 2HCI.

In the present disclosure, the term “STING” is an abbreviation for “Stimulator of Interferon Genes”, and the “STING modulator” refers to a substance that activates or inhibits STING receptors in vitro or in vivo. The STING modulator includes but is not limited to a cyclic dinucleotide (CDN) or an acyclic dinucleotide agonist.

Unless otherwise specified, the experimental methods, detection methods and preparation methods disclosure in the present disclosure all adopt conventional techniques in molecular biology, biochemistry, cell culture and related fields conventional in the art. These techniques have been well described in the existing literature. For details, please refer to Sambrook et al. MOLECULAR CLONING: A LABORATORY MANUAL, Second edition, Cold Spring Harbor Laboratory Press, 1989 and Third edition, 2001; Ausubel et al. CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, Chromatin (P. M. Wassarman and A. P. Wolffe, eds.), Academic Press, San Diego, 1999; and METHODS IN MOLECULAR BIOLOGY, Vol. 119, Chromatin Protocols (P. B. Becker, ed.) Humana Press, Totowa, 1999, etc.

Unless otherwise specified, the materials, reagents, etc. used in the present disclosure can be obtained commercially or synthesized according to known methods, unless otherwise specified.

### Example 1 Effect of Treating Neutrophils with Different Concentrations of Single Inducers on Neutrophil Viability

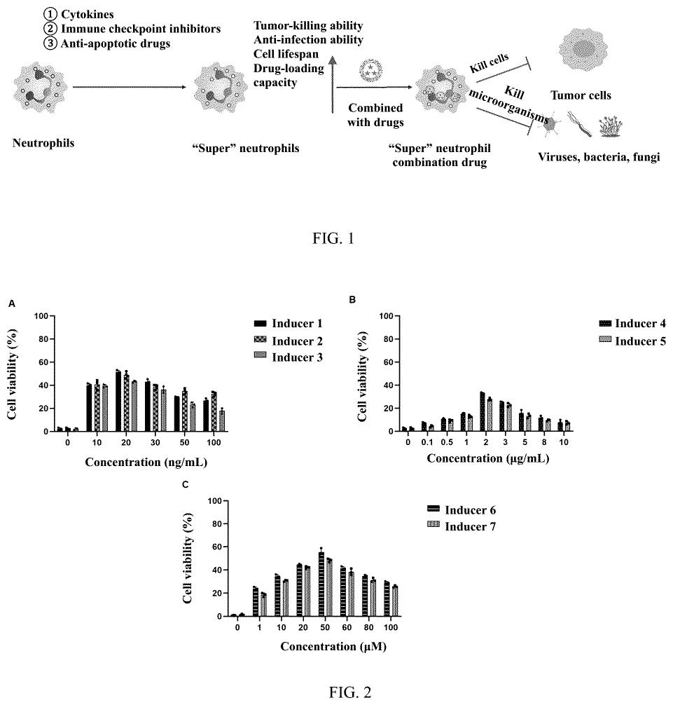

Human neutrophils were incubated with different concentrations of single inducers in 1640 medium for 8 h. Different concentrations of single inducers included:
inducer 1 (IFNγ): 0, 10, 20, 30, 50, 100 ng/mL;
inducer 2 (GM-CSF): 0, 10, 20, 30, 50, 100 ng/mL;
inducer 3 (TNFα): 0, 10, 20, 30, 50, 100 ng/mL;
inducer 4 (duvalumab): 0.1, 0.5, 1, 2, 3, 5, 8, 10 μg/mL;
inducer 5 (avelumab): 0.1, 0.5, 1, 2, 3, 5, 8, 10 μg/mL;
inducer 6 (Q-VD-Oph): 1, 10, 20, 50, 60, 80, 100 μM; and
inducer 7 (Nec-1): 1, 10, 20, 50, 60, 80, 100 μM; and
conditions of culture were 37° C. and 5% CO₂. The apoptosis of neutrophils was detected by flow cytometry after removing the inducer and continuing culture for 24 h. The cell viability results are shown in FIG. 2.

The results in FIG. 2 showed that among inducers 1-3, all of which were cytokines, both inducers 1 and 2 can significantly prolong the lifespan of neutrophils and had better effects than inducer 3, with the best effect observed at a concentration of 20 ng/mL. Among inducers 4-5, both of which were PD-L1 inhibitors, both can increase neutrophil viability, with the best effect observed at 2 μg/mL. Among inducers 6-7, both of which were apoptosis inhibitors, both can increase neutrophil viability, with the best effect observed at 50 μM.

### Example 2 Demonstration that Treatment with Combined Inducers can Enhance Neutrophil Viability

The components of combined inducer 1 were: 20 ng/mL IFNγ+20 ng/mL GM-CSF.

The components of combined inducer 2 were: 2 μg/mL durvalumab+50 μM Q-VD-Oph.

The components of combined inducer 3 were: 20 ng/mL IFNγ+2 μg/mL durvalumab+50 μM Q-VD-Oph.

The components of combined inducer 4 were: 20 ng/mL GM-CSF+2 μg/mL durvalumab+50 μM Q-VD-Oph.

The components of combined inducer 5 were: 20 ng/mL IFNγ+20 ng/mL GM-CSF+2 μg/mL durvalumab+50 μM Q-VD-Oph.

The components of combined inducer 6 were: 20 ng/mL IFNγ+50 μM Q-VD-Oph. Human neutrophils were incubated with different combined inducers in 1640 medium for 8 h under conditions of culture of 37° C. and 5% CO₂. The apoptosis of neutrophils was detected by flow cytometry after removing the inducer and continuing culture for 24 h. The cell viability results are shown in FIG. 3.

FIG. 3 showed that neutrophils treated with combined inducer 3 had a longer in vitro lifespan. In particular, neutrophils can survive for at least 24 h in vitro after removing the inducer with cell viability above 90%.

### Example 3 Demonstration that Treatment with Combined Inducers can Enhance the Tumor-Killing Ability of Neutrophils

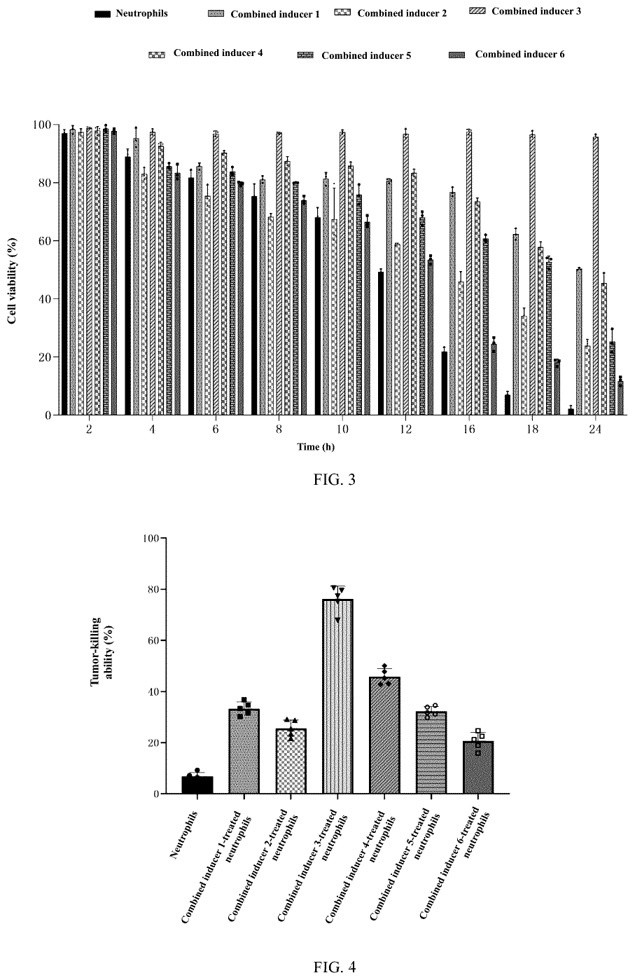

Human neutrophils were incubated with the above different combined inducers in 1640 medium for 8 h under conditions of culture of 37° C. and 5% CO₂. Subsequently, PBS was used for washing three times, centrifugation was performed at 1800 rpm and 4° C. for 3 min, and the supernatant was discarded to obtain “super” neutrophils. The “super” neutrophils were incubated with human breast cancer cell MCF7 at an effector-target ratio of 5:1 at 37° C. and 5% CO₂ for 24 h, where all MCF7 were transfected with lentiviruses to stably express mcherry. The fluorescence of mcherry was detected by a microplate reader after washing twice with PBS. The tumor-killing ability was calculated according to the following method: [(experimental well fluorescence value−blank well fluorescence value)/(control well fluorescence value−blank well fluorescence value)]×100%. The results are shown in FIG. 4.

FIG. 4 showed that combined inducer 3 can more significantly enhance the tumor-killing ability of neutrophils.

### Example 4 Demonstration that a Combined Inducer was More Effective in Enhancing Neutrophil Viability than Single Inducers

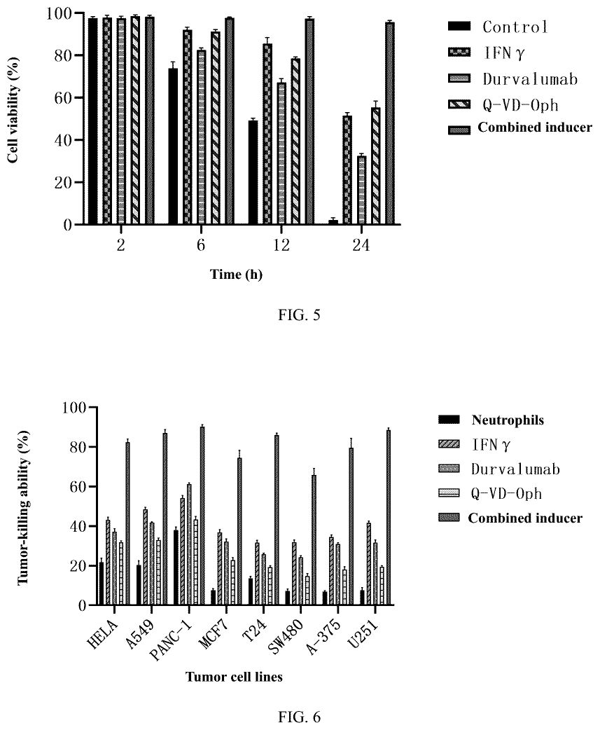

Human neutrophils were incubated in 1640 medium containing 20 ng/mL IFNγ, or 2 μg/mL durvalumab, or 50 μM Q-VD-Oph, or a combined inducer containing the three for 8 h under conditions of culture of 37° C. and 5% CO₂. The apoptosis of neutrophils was detected by flow cytometry after removing the inducer and continuing culture for 24 h. The detection results are shown in FIG. 5.

FIG. 5 showed that neutrophils treated with the combined inducer had a longer in vitro lifespan compared with those treated with the single inducers, and can survive for at least 24 h in vitro with 90% cell viability.

### Example 5 Demonstration that Neutrophils Treated with a Combined Inducer had a Stronger Tumor-Killing Ability than Those Treated with Single Inducers

Human neutrophils were incubated in 1640 medium containing 20 ng/mL IFNγ, or 2 μg/mL durvalumab, or 50 μM Q-VD-Oph, or a combined inducer containing the three for 8 h under conditions of culture of 37° C. and 5% CO₂. Subsequently, PBS was used for washing three times, centrifugation was performed at 1800 rpm and 4° C. for 3 min, and the supernatant was discarded to obtain “super” neutrophils. The “super” neutrophils were incubated with various tumor cells at an effector-target ratio of 5:1 at 37° C. and 5% CO₂ for 24 h, where all the tumor cells were transfected with lentiviruses to stably express mcherry. The fluorescence of mcherry was detected by a microplate reader after washing twice with PBS. The tumor-killing ability was calculated according to the following method: [(experimental well fluorescence value−blank well fluorescence value)/(control well fluorescence value−blank well fluorescence value)]×100%. The detection results are shown in FIG. 6. FIG. 6 showed that neutrophils treated with the combined inducer had a stronger tumor-killing ability compared with those treated with the single inducers.

### Example 6 Demonstration that the Present Construction Method can Remodel Neutrophils from Different Sources into Neutrophils with a Stronger Anti-Tumor Ability

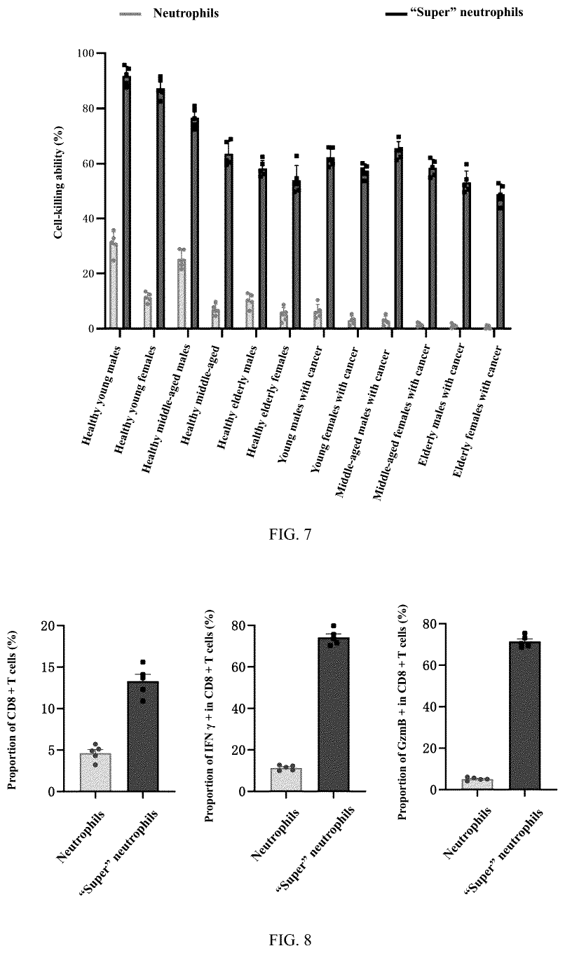

Blood from volunteers of different ages and health states was taken, and neutrophils were obtained by density gradient centrifugation. Human neutrophils were incubated in 1640 medium containing a combined inducer (20 ng/mL IFNγ+2 μg/mL durvalumab+50 μM Q-VD-Oph) for 8 h under conditions of culture of 37° C. and 5% CO₂. Subsequently, PBS was used for washing three times, centrifugation was performed at 1800 rpm and 4° C. for 3 min, and the supernatant was discarded to obtain “super” neutrophils. The “super” neutrophils were incubated with various tumor cells at an effector-target ratio of 5:1 at 37° C. and 5% CO₂ for 24 h, where all the tumor cells were transfected with lentiviruses to stably express mcherry. The fluorescence of mcherry was detected by a microplate reader after washing twice with PBS. The tumor-killing ability was calculated according to the following method: [(experimental well fluorescence value−blank well fluorescence value)/(control well fluorescence value−blank well fluorescence value)]×100%. The detection results are shown in FIG. 7. FIG. 7 showed that the present construction method can enhance the tumor-killing ability of neutrophils from different sources.

### Example 7 Demonstration that “Super” Neutrophils can Effectively Activate the Body's Immune Response

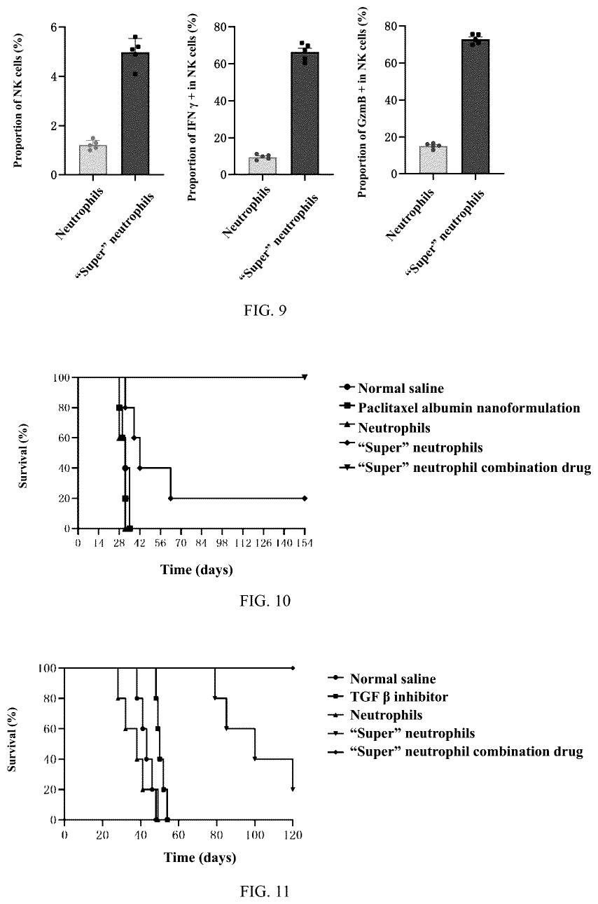

Mouse models of orthotopic breast cancer were constructed, and 5×10⁶ neutrophils or “super neutrophils” induced by 20 ng/mL IFNγ+2 μg/mL durvalumab+50 μM Q-VD-Oph were injected into the mice via the tail vein. After 24 h, the tumor tissue of the mice was stripped, washed with Hanks' buffer, cut into small pieces with a volume of less than 1 mm³ with scissors, digested with 0.2% type IV collagenase at 37° C. for 6 h, added DMEM medium containing 10% serum to terminate the digestion, and passed through a cell filter to obtain cells. After the cells were resuspended in Hanks' buffer and washed three times, flow antibody staining was performed, the proportions of T cells and NK cells at the tumor site and the proportions of T cells and NK cells with high tumor-killing activity (IFNγ+, GzmB+) in T cells and NK cells were analyzed by flow cytometry. The respective results are shown in FIG. 8 and FIG. 9. FIGS. 8-9 indicated that compared to untreated neutrophils, “super” neutrophils had significantly stronger recruitment and activation effects on T cells and NK cells.

### Example 8 Demonstration that a “Super” Neutrophil Combination Drug can Synergistically Treat Gliomas

1×10⁵/mL super neutrophils induced with the combined inducer (20 ng/mL IFNγ+2 μg/mL durvalumab+50 μM Q-VD-Oph) prepared according to Example 2 were mixed with 50 μg/mL paclitaxel albumin nanoformulation, incubated at 37° C. and 5% CO₂ for 50 min, followed by centrifugation at 1800 rpm and 4° C. for 3 min, and the supernatant was discarded to obtain a “super” neutrophil combination drug. Mouse glioma models were constructed, and normal saline (200 μL), paclitaxel albumin nanoformulation (10 mg/kg, 200 μL), neutrophils (5×10⁶, 200 μL), “super” neutrophils (5×10⁶, 200 μL), and “super” neutrophil combination drug (5×10⁶, 200 μL) were injected into the mice via the tail vein respectively. The results are shown in FIG. 10. The results in FIG. 10 indicated that the “super” neutrophil combination drug can significantly prolong the survival of mice and had a better therapeutic effect on gliomas.

### Embodiment 9 Demonstration that a “Super” Neutrophil Combination Drug can Synergistically Treat Lung Metastasis of Triple-Negative Breast Cancer

6×10⁶/mL super neutrophils induced with the combined inducer (20 ng/mL IFNγ+2 g/mL durvalumab+50 μM Q-VD-Oph) prepared according to Example 2 were mixed with g/mL TGFβ inhibitor SB525334 liposomes, incubated at 37° C. and 5% CO₂ for 50 min, followed by centrifugation at 1800 rpm and 4° C. for 3 min, and the supernatant was discarded to obtain a “super” neutrophil combination drug. Mouse models of triple-negative breast cancer with spontaneous lung metastasis were constructed, and normal saline (200 μL), TGFβ inhibitor liposomes (1 mg/kg, 200 μL), neutrophils (6×10⁶, 200 μL), “super” neutrophils (6×10⁶, 200 μL), and “super” neutrophil combination drug (6×10⁶, 200 μL) were injected into the mice via the tail vein respectively. The results are shown in FIG. 11. FIG. 11 indicated that the “super” neutrophil combination drug can significantly prolong the survival of mice and had a better therapeutic effect on lung metastasis of breast cancer.

The “super” neutrophil combination drug of the present disclosure can effectively treat in orthotopic tumors and metastatic tumors, and can exert a better tumor therapeutic effect compared with free drugs, untreated neutrophils and pure “super” neutrophils.

### Example 10 Demonstration that a “Super” Neutrophil Combination Drug can Synergistically Treat Fungal Infections

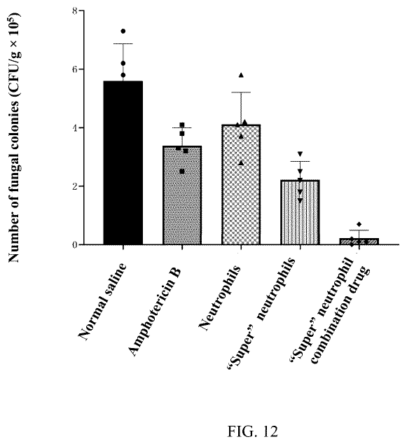

1.2×10⁷/mL super neutrophils induced with the combined inducer (20 ng/mL IFNγ+2 μg/mL durvalumab+50 μM Q-VD-Oph) prepared according to Example 2 were mixed with 2 μg amphotericin B nanoparticles, incubated at 37° C. and 5% CO₂ for 50 min, followed by centrifugation at 1800 rpm and 4° C. for 3 min, and the supernatant was discarded to obtain a “super” neutrophil combination drug. Mouse fungal infection models were constructed, namely, after chemotherapy, C57BL6 mice were injected with 1×10⁵ Cryptococcus neoformans (H99) via the tail vein, and after 3 h, normal saline (250 μL), amphotericin B (2 μg/mL, 250 μL), neutrophils (3×10⁶/mL, 250 μL), “super” neutrophils (3×10⁶, 250 μL), and “super” neutrophil combination drug (3×10⁶, 250 μL) were injected into the mice via the tail vein respectively. The results are shown in FIG. 12. The results in FIG. 12 indicated that the “super” neutrophil combination drug can significantly reduce the bacterial count in brain tissue and had a better anti-infective effect.

It should be understood that all the above examples are exemplary and not intended to encompass all possible embodiments included in the claims. Various modifications and changes can be made on the basis of the above examples without departing from the scope of the present disclosure. Similarly, various technical features of the above examples can be combined arbitrarily to form additional examples of the present disclosure that may not be explicitly described. Therefore, the above examples only express several examples of the present disclosure and do not limit the scope of protection of the patent of the present disclosure.

1. A construction method for obtaining an enhanced neutrophil, comprising: performing induction culture of a neutrophil in a medium comprising any one or more of component A, component B and component C, wherein
the component A comprises one or more of IFNγ, GM-CSF, G-CSF, IFN-β, IFN-α, TNFα, LPS, IL1β, IL6, MCP-1, IL-23, BCG, or β-glucan;
the component B comprises one or more of durvalumab, avelumab, atezolizumab, ipilimumab, or tremelimumab; and
the component C comprises one or more of Q-VD-Oph, DFO, Hsp70, NAC, or Nec-1.

2. The construction method according to claim 1, wherein the component A is selected from IFNγ and/or GM-CSF;
preferably, the component B is selected from durvalumab;
preferably, the component C is selected from Q-VD-Oph;
preferably, a concentration of the component A is 1-200 ng/mL, preferably 1-100 ng/mL, more preferably 1-50 ng/mL, particularly preferably 1-30 ng/mL, and most preferably 20 ng/mL;
preferably, a concentration of the component B is 0.1-10 μg/mL, preferably 0.5-8 μg/mL, more preferably 0.5-5 μg/mL, particularly preferably 1-3 μg/mL, and most preferably 2 μg/mL; and
preferably, a concentration of the component C is 1-100 μM, preferably 10-80 μM, more preferably 10-60 μM, particularly preferably 20-60 μM, and most preferably 50 μM.

3. The construction method according to claim 1, wherein the medium is 1640 medium or DMEM medium.

4. The construction method according to claim 1, wherein the construction method comprises: incubating the neutrophil in the medium in an aerobic or anaerobic environment, preferably, a temperature of the incubating is controlled at 35-40° C., preferably 36-40° C., and more preferably 37° C.;
preferably, time of the incubating is controlled to be 2-48 h, preferably 2-36 h, more preferably 4-24 h, and most preferably 4-18 h;
preferably, the incubating is controlled to be carried out under a condition of 5% CO₂; and
preferably, the neutrophil is derived from a healthy individual or a tumor patient.

5. An enhanced neutrophil obtained by the construction method according to claim 1.

6. A drug composition, comprising the enhanced neutrophil obtained by the construction method according to claim 1.

7. The drug composition according to claim 6, wherein the drug composition further comprises a nano-drug;
preferably, the nano-drug is selected from an anti-tumor drug, an immunomodulatory drug and an antibacterial drug;
preferably, the anti-tumor drug is selected from paclitaxel, doxorubicin, 5-fluorouracil, and camptothecin;
preferably, the immunomodulatory drug is selected from a STING modulator, a TGF-β modulator and a HIF-1α modulator; and
preferably, the antibacterial drug is selected from amphotericin B, colistin, azithromycin, clavulanic acid, ampicillin, ciprofloxacin, and vancomycin.

8. A drug composition, comprising the enhanced neutrophil according to claim 5.

9. The drug composition according to claim 8, wherein the drug composition further comprises a nano-drug;
preferably, the nano-drug is selected from an anti-tumor drug, an immunomodulatory drug and an antibacterial drug;
preferably, the anti-tumor drug is selected from paclitaxel, doxorubicin, 5-fluorouracil, and camptothecin;
preferably, the immunomodulatory drug is selected from a STING modulator, a TGF-β modulator and a HIF-la modulator; and
preferably, the antibacterial drug is selected from amphotericin B, colistin, azithromycin, clavulanic acid, ampicillin, ciprofloxacin, and vancomycin.

10. A method for preparing a drug composition, comprising: mixing the enhanced neutrophil obtained by the construction method according to claim 1 with a nano-drug, and then incubating in an aerobic or anaerobic environment.

11. The preparation method according to claim 10, wherein a temperature of the incubating is controlled at 35-40° C., preferably 36-40° C., and more preferably 37° C.;
preferably, time of the incubating is controlled to be 0.1-12 h, preferably 0.2-10 h, more preferably 0.2-6 h, and most preferably 0.2-3 h; and
preferably, the incubating is controlled to be carried out under a condition of 5% CO₂.

12. A method for preparing a drug composition, comprising: mixing the enhanced neutrophil according to claim 5 with a nano-drug, and then incubating in an aerobic or anaerobic environment.

13. The preparation method according to claim 12, wherein a temperature of the incubating is controlled at 35-40° C., preferably 36-40° C., and more preferably 37° C.;
preferably, time of the incubating is controlled to be 0.1-12 h, preferably 0.2-10 h, more preferably 0.2-6 h, and most preferably 0.2-3 h; and
preferably, the incubating is controlled to be carried out under a condition of 5% CO₂.

14. An application of the enhanced neutrophil obtained by the construction method according to claim 1 in preventing and/or treating of a tumor, an infection or an immune disease.

15. An application of the enhanced neutrophil according to claim 5 in preventing and/or treating of a tumor, an infection or an immune disease.

16. An application of the drug composition according to claim 6 in preventing and/or treating of a tumor, an infection or an immune disease.

17. An application of the drug composition according to claim 8 in preventing and/or treating of a tumor, an infection or an immune disease.

18. An application of the drug composition obtained by the preparation method according to claim 10 in preventing and/or treating of a tumor, an infection or an immune disease.

19. An application of the drug composition obtained by the preparation method according to claim 12 in preventing and/or treating of a tumor, an infection or an immune disease.
